(19) United States
(12) Patent Application Publication
(10) Pub. No.: US 2020/0237773 A1
(43) Pub. Date: Jul. 30, 2020

(54) THERAPEUTIC COMBINATION OF A THIRD-GENERATION EGFR TYROSINE KINASE INHIBITOR AND A RAF INHIBITOR

(71) Applicant: Novartis AG, Basel (CH)

(72) Inventors: Susan Moody, Cambridge, MA (US); Lilli Petruzzelli, Cambridge, MA (US); Jeffrey Engelman, Cambridge, MA (US)

(21) Appl. No.: 16/634,924

(22) PCT Filed: Aug. 1, 2018
(86) PCT No.: PCT/IB2018/055792

§ 371 Date: Jan. 29, 2020

Related U.S. Application Data
(60) Provisional application No. 62/540,595, filed on Aug. 3, 2017.

(51) Int. Cl. A61K 31/55; A61K 31/5377; A61P 35/00

(52) U.S. Cl. CPC A61K 31/55; A61P 35/00; A61K 31/5377

(57) ABSTRACT

This invention relates to a pharmaceutical combination comprising (a) a third generation EGFR tyrosine kinase inhibitor and (b) a Raf inhibitor, particularly for use in the treatment of a cancer, particularly a lung cancer. This invention also relates to uses of such a combination for the preparation of a medicament for the treatment of a cancer; methods of treating a cancer in a subject in need thereof comprising administering to said subject a jointly therapeutically effective amount of said combination; pharmaceutical compositions comprising such combination and commercial packages thereto.

### FIELD OF THE INVENTION

The present invention relates to a method of treating a cancer, e.g. lung cancer, in particular non-small cell lung cancer (NSCLC), in a human subject and to pharmaceutical combinations useful in such treatment. In particular, the present invention provides a pharmaceutical combination comprising (a) a third-generation EGFR tyrosine kinase inhibitor (TKI), particularly (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof, and (b) a Raf inhibitor, particularly N-(3-(2-(2-hydroxyethoxy)-6-morpholinopyridin-4-yl)-4-methylphenyl)-2-(trifluoromethyl)isonicotinamide, or a pharmaceutically acceptable salt thereof. There is also provided such combinations for use in the treatment of a cancer, in particular a lung cancer (e.g. NSCLC); the use of such combinations for the preparation of a medicament for the treatment of a cancer, in particular a lung cancer (e.g. NSCLC); methods of treating a cancer, in particular a lung cancer (e.g. NSCLC), in a human subject in need thereof comprising administering to said subject a jointly therapeutically effective amount of said combinations; pharmaceutical compositions comprising such combinations and commercial packages thereto.

### BACKGROUND ART

Lung cancer is the most common and deadly cancer worldwide, with non-small cell lung cancer (NSCLC) accounting for approximately 85% of lung cancer cases. In Western countries, 10-15% non-small cell lung cancer (NSCLC) patients express epidermal growth factor receptor (EGFR) mutations in their tumors and Asian countries have reported rates as high as 30-40%. The predominant oncogenic EGFR mutations (L858R and ex19del) account for about 85% of EGFR NSCLC.

EGFR-mutant patients are given an EFGR inhibitor as first line therapy. However, most patients develop acquired resistance, generally within 10 to 14 months. In up to 50% of NSCLC patients harboring a primary EGFR mutation treated with first generation reversible EGFR tyrosine kinase inhibitors (TKIs), also referred to as first-generation TKIs, such as erlotinib, gefitinib and icotinib, a secondary “gatekeeper” T790M mutation develops.

Second-generation EGFR TKIs (such as afatinib and dacomitinib) have been developed to try to overcome this mechanism of resistance. These are irreversible agents that covalently bind to cysteine 797 at the EGFR ATP site. Second generation EGFR TKIs are potent on both activating [L858R, ex19del] and acquired T790M mutations in pre-clinical models. Their clinical efficacy has however proven to be limited, possibly due to severe adverse effects caused by concomitant wild-type (WT) EGFR inhibition. Resistance to second-generation inhibitors also soon develops, with virtually all patients receiving first- and second-generation TKIs becoming resistant after approximately 9-13 months.

This has led to the development of third-generation EGFR TKIs, e.g. nazartinib (EGF816), rociletinib, ASP8273 and osimertinib (Tagrisso®). Third-generation EGFR TKIs are WT EGFR sparing and also have relative equal potency for activating EGFR mutations [such as L858R and ex19del] and acquired T790M. Osimertinib has recently been approved in the United States for the treatment of patients with advanced EGFR T790M+NSCLC whose disease has progressed on or after an EGFR TKI therapy.

However, resistance to these third generation agents also soon develops. There are multiple mechanisms which are thought to give rise to acquired resistance to third-generation EGFR TKIs; these mechanisms are also less well-characterized. In some cases, resistance has been found to be associated with amplification of MET or FGFR1, or mutation of BRAF (Ho et al, (Journal of Thoracic Oncology, 2016) or a tertiary EGFR C797S mutation, which was found in the plasma sample of a patient progressing on osimertinib treatment (Thress et al (Nature Medicine, 21(6), 2015, pp 560-562).

Thus there remains a need for therapeutic options to prevent or delay the emergence of resistance (e.g., by inducing more durable remissions) in the course of treatment with EGFR tyrosine kinase inhibitors (TKIs), particularly third generation EGFR TKIs; and/or overcome or reverse resistance acquired in the course of treatment with EGFR tyrosine kinase inhibitors, particularly third generation EGFR TKIs. There also remains a continued need to develop new treatment options in NSCLC, particularly EGFR mutant NSCLC, as the disease remains incurable despite the efficacy of EGFR TKIs.

### SUMMARY OF THE INVENTION

The present inventors have found that the combination of Compound B, a compound of formula (II) below, to a third-generation EGFR TKI such as nazartinib prolonged and deepened the response to the third-generation EGFR TKI as single agent. This opens up the possibility of an effective therapeutic option in this clinical setting, where no effective therapy currently exists.

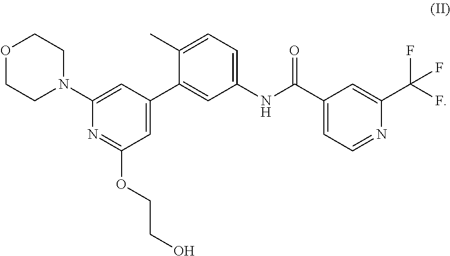 [SMILES: Cc1ccc(NC(=O)c2ccnc(C(F)(F)F)c2)cc1-c1cc(OCCO)nc(N2CCOCC2)c1]

An object of the present invention is therefore to provide a therapy to improve the treatment of a cancer, particularly non-small cell lung cancer, more particularly EGFR-mutant NSCLC. In particular, the aim of the present invention is to provide a safe and tolerable treatment which deepens the initial response and/or prevents or delays the emergence of drug resistance, particularly resistance to EGFR TKI therapy. The pharmaceutical combinations described herein are expected to be safe and tolerable and also improve the depth and/or duration of response to EGF816 in treatment-naive and/or third generation EGFR-TKI naive, T790M+EGFR-mutant NSCLC, including T790M+EGFR-mutant advanced NSCLC.

The present invention provides a pharmaceutical combination comprising (a) a third-generation EGFR tyrosine kinase inhibitor and (b) a Raf inhibitor, as one aspect of the invention.

The present invention also provides a pharmaceutical combination comprising (a) the compound of formula I

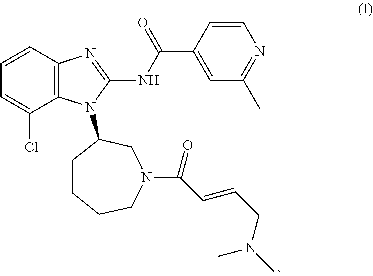 [SMILES: Cc1cc(C(=O)Nc2nc3cccc(Cl)c3n2[C@@H]2CCCCN(C(=O)/C=C/CN(C)C)C2)ccn1]

which is also known as (R,E)-N-(7-chloro-1-(1-(4-(dimethylamino)but-2-enoyl)azepan-3-yl)-1H-benzo[d]imidazol-2-yl)-2-methylisonicotinamide (referred to herein as “Compound A”), or a pharmaceutically acceptable salt thereof, and (b) a Raf inhibitor.

In a preferred aspect, the present invention also relates to a pharmaceutical combination, referred to as a COMBINATION OF THE INVENTION, comprising (a) a compound which is the compound of formula I below

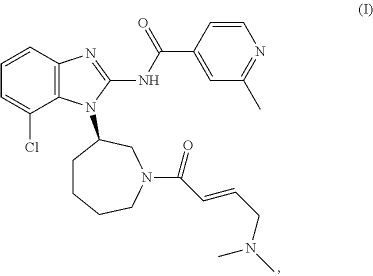 [SMILES: Cc1cc(C(=O)Nc2nc3cccc(Cl)c3n2[C@@H]2CCCCN(C(=O)/C=C/CN(C)C)C2)ccn1]

which is also known as (R,E)-N-(7-chloro-1-(1-(4-(dimethylamino)but-2-enoyl)azepan-3-yl)-1H-benzo[d]imidazol-2-yl)-2-methylisonicotinamide (also referred to herein as “Compound A”), or a pharmaceutically acceptable salt thereof, and (b) a compound of formula (II),

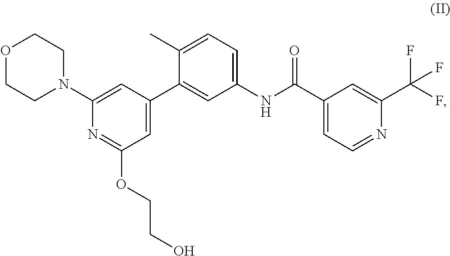 [SMILES: Cc1ccc(NC(=O)c2ccnc(C(F)(F)F)c2)cc1-c1cc(OCCO)nc(N2CCOCC2)c1]

or a pharmaceutically acceptable salt thereof.

In another aspect, the present invention relates to a dosing regimen suitable for the administration of a third generation EGFR tyrosine kinase inhibitor in combination with a Raf inhibitor. The present invention provides a therapeutic regimen which maximizes the therapeutic efficacy of a third generation EGFR tyrosine kinase inhibitor (TKI) in the early stages of EGFR TKI cancer therapy followed by the administration of a pharmaceutical combination of a third generation EGFR TKI and a Raf inhibitor during the period of relatively stable disease control which follows, when the tumor is in a state of minimal residual disease.

It is envisaged that the therapeutic agents of the present invention may be usefully administered according to a dosing regimen which involves the administration of the third generation EGFR tyrosine kinase inhibitor, e.g. Compound A, or a pharmaceutically acceptable salt thereof, as a single agent for a period of time sufficient to achieve relatively stable disease control (i.e., a state of minimal residual disease), followed by the administration of the combination of Compound A, or a pharmaceutically acceptable salt thereof, and a Raf inhibitor, particularly, Compound B or a pharmaceutically acceptable salt thereof.

The present invention therefore provides a method for treating EGFR mutant lung cancer in a human in need thereof, particularly EGFR mutant NSCLC, comprising

(a) administering a therapeutically effective amount of a third-generation EFGR tyrosine kinase inhibitor (e.g. Compound A, or a pharmaceutically acceptable salt thereof) as monotherapy until minimal residual disease is achieved (i.e., the tumor burden decrease is less than 5% between two assessments carried out at least one month apart); followed by

(b) administering a therapeutically effective amount of a pharmaceutical combination of said third-generation EGFR tyrosine kinase inhibitor (e.g. Compound A, or a pharmaceutically acceptable salt thereof), and a Raf inhibitor, particularly, Compound B or a pharmaceutically acceptable salt thereof.

The present invention provides a third-generation EFGR tyrosine kinase inhibitor (such as Compound A, or a pharmaceutically acceptable salt thereof), for use in treating EGFR mutant lung cancer in a human in need thereof, particularly EGFR mutant NSCLC, wherein

(a) the third-generation EGFR tyrosine kinase inhibitor (such as Compound A, or a pharmaceutically acceptable salt thereof) is administered as monotherapy until minimal residual disease is achieved (i.e., the tumor burden decrease is less than 5% between two assessments carried out at least one month apart); and

(b) a pharmaceutical combination of the third-generation EGFR tyrosine kinase inhibitor (such as Compound A, or a pharmaceutically acceptable salt thereof), and a Raf inhibitor, particularly, Compound B or a pharmaceutically acceptable salt thereof, is thereafter administered.

In another aspect, the present invention relates to the COMBINATION OF THE INVENTION for simultaneous, separate or sequential use.

In another aspect, the present invention relates to the COMBINATION OF THE INVENTION for use in the treatment of a cancer, particularly non-small cell lung cancer, more particularly EGFR mutant NSCLC.

In another aspect, the present invention relates to a method of treating a cancer, particularly non-small cell lung cancer, more particularly EGFR mutant NSCLC, comprising simultaneously, separately or sequentially administering to a subject in need thereof the COMBINATION OF THE INVENTION in a quantity which is jointly therapeutically effective against said cancer.

In another aspect, the present invention relates to the use of the COMBINATION OF THE INVENTION for the preparation of a medicament for the treatment of a cancer, particularly non-small cell lung cancer, more particularly EGFR mutant NSCLC.

The present invention also provides a third generation EGFR tyrosine kinase inhibitor, particularly (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof, for use in a combination therapy with a Raf inhibitor, particularly N-(3-(2-(2-hydroxyethoxy)-6-morpholinopyridin-4-yl)-4-methylphenyl)-2-(trifluoromethyl)isonicotinamide, or a pharmaceutically acceptable salt thereof, or a pharmaceutically acceptable salt thereof, for the treatment of a cancer, in particular a lung cancer (e.g. NSCLC).

A Raf inhibitor, particularly N-(3-(2-(2-hydroxyethoxy)-6-morpholinopyridin-4-yl)-4-methylphenyl)-2-(trifluoromethypisonicotinamide, or a pharmaceutically acceptable salt thereof, for use in a combination therapy with a third generation EGFR tyrosine kinase inhibitor, particularly (R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof, for the treatment of a cancer, in particular a lung cancer (e.g. NSCLC) is also provided.

### DETAILED DESCRIPTION OF THE FIGURES

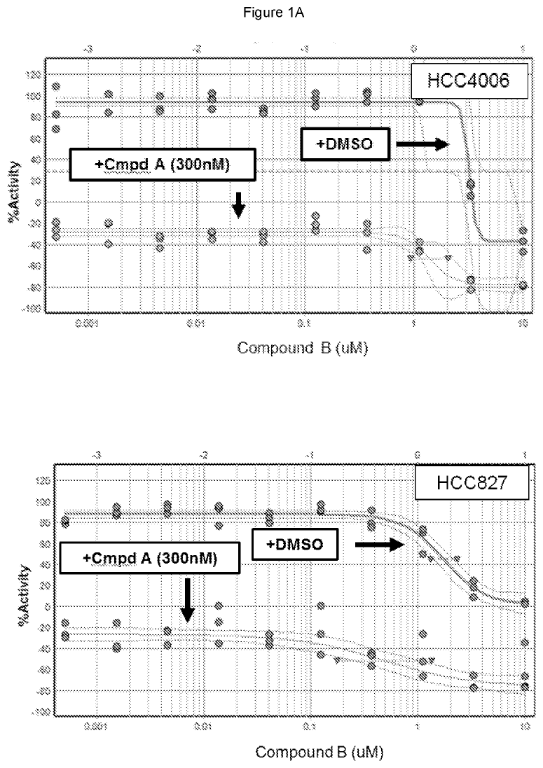

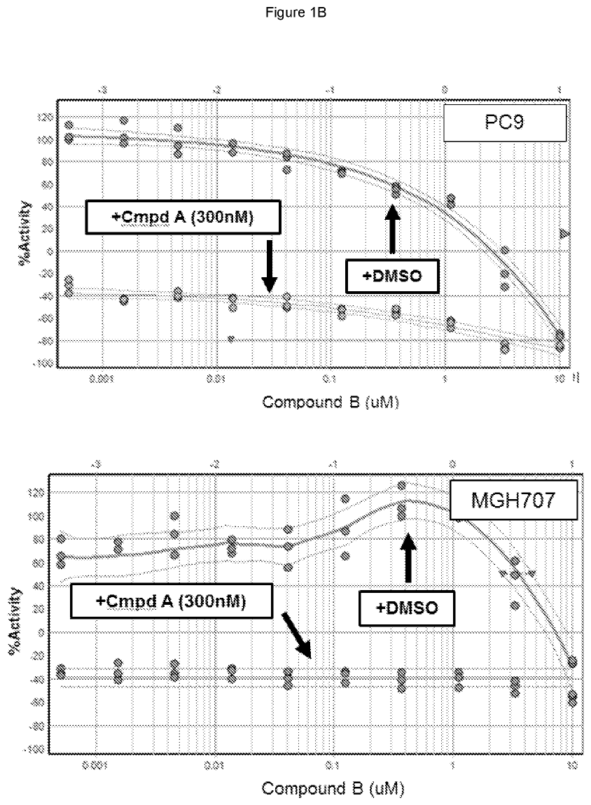

FIG. 1A and FIG. 1B: Dose response curves in EGFR mutant NSCLC cell lines (FIG. 1A: HCC4006 and HCC827 cell lines. FIG. 1B: PC9 and MGH707 cell lines). for Compound B in the presence of DMSO (curves at the top of Figures) or 300nM EGF816 (“Cmpd A”-curves at the bottom of Figures). % Activity is a measure of cell number as read out by cell-titer glo. 0 represents the CTG value at Day 0 and 100 represents the value of untreated growth at day 5.

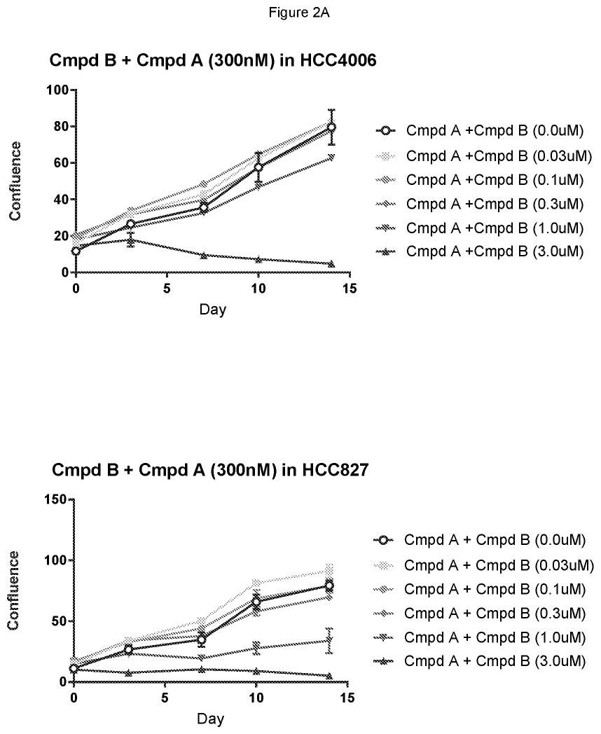

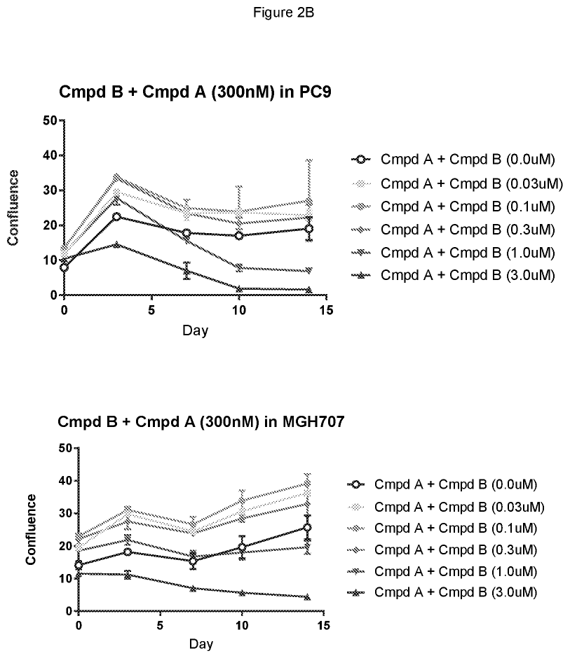

FIG. 2A and FIG. 2B: Dose response of Compound B (“Cmpd B”) in EGFR mutant NSCLC lines in combination with Compound A (EGF816) (“Cmpd A”) (300 nM) (FIG. 2A: HCC4006 and HCC827 cell lines. FIG. 2B: PC9 and MGH707 cell lines). Cells were treated with fresh drug and imaged for confluence measurement twice per week for two weeks. Cell confluence was used as a surrogate for cell number. It is shown that a Raf-inhibitor in combination with EGF816 slows drug-tolerant cell outgrowth.

### DETAILED DESCRIPTION OF THE INVENTION

In one aspect, the present invention relates to a pharmaceutical combination comprising a third-generation EGFR tyrosine kinase inhibitor and a Raf inhibitor. This pharmaceutical combination is hereby referred to as “COMBINATION OF THE INVENTION”.

The present invention also relates to a pharmaceutical combination comprising (a) a compound of formula (I)

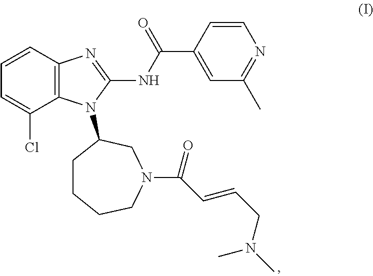 [SMILES: Cc1cc(C(=O)Nc2nc3cccc(Cl)c3n2[C@@H]2CCCCN(C(=O)/C=C/CN(C)C)C2)ccn1]

which is also known as (R,E)-N-(7-chloro-1-(1-(4-(dimethylamino)but-2-enoyl)azepan-3-yl)-1H-benzo[d]imidazol-2-yl)-2-methylisonicotinamide (also herein referred to as “Compound A”), or a pharmaceutically acceptable salt thereof, and (b) a Raf inhibitor.

In a preferred aspect, the present invention also relates to a pharmaceutical combination, which is also referred to as a COMBINATION OF THE INVENTION, comprising (a) a compound which is the compound of formula I below

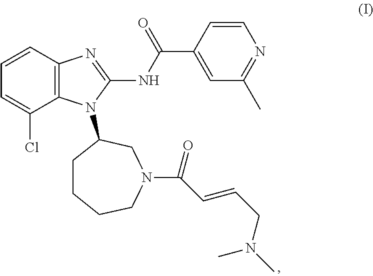 [SMILES: Cc1cc(C(=O)Nc2nc3cccc(Cl)c3n2[C@@H]2CCCCN(C(=O)/C=C/CN(C)C)C2)ccn1]

which is also known as (R,E)-N-(7-chloro-1-(1-(4-(dimethylamino)but-2-enoyl)azepan-3-yl)-1H-benzo[d]imidazol-2-yl)-2-methylisonicotinamide (also referred to herein as “Compound A”), or a pharmaceutically acceptable salt thereof, and (b) a compound of formula (II),

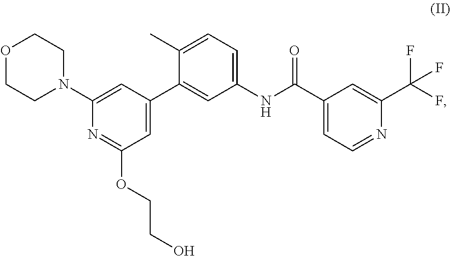 [SMILES: Cc1ccc(NC(=O)c2ccnc(C(F)(F)F)c2)cc1-c1cc(OCCO)nc(N2CCOCC2)c1]

or a pharmaceutically acceptable salt thereof.

### Third-Generation EGFR Tyrosine Kinase Inhibitors

Third-generation EGFR TKIs are wild-type (WT) EGFR sparing and also have relative equal potency for activating EGFR mutations [such as L858R and ex19del] and acquired T790M.

The preferred third generation EGFR inhibitor which is used in the present combinations and the preferred dosages described herein is Compound A, also known as nazartinib and as “EGF816”. Compound A is a targeted covalent irreversible inhibitor of Epidermal Growth Factor Receptor (EGFR) that selectively inhibits activating and acquired resistance mutants (L858R, ex19del and T790M), while sparing wild-type (WT) EGFR (see Jia et al, Cancer Res Oct. 1, 2014 74; 1734). Compound A has shown significant efficacy in EGFR mutant (L858R, ex19del and T790M) cancer models (in vitro and in vivo) with no indication of WT EGFR inhibition at clinically relevant efficacious concentrations. Dose-dependent anti-tumor efficacy was observed in several xenograft models and Compound A was well tolerated with no body weight loss observed at efficacious doses.

Compound A was found to show durable antitumor activity in a clinical study with patients suffering from advanced non-small cell lung cancer (NSCLC) harboring T790M (see Tan et al, Journal of Clinical Oncology 34, no. 15_suppl (May 2016)).

Pharmaceutical compositions comprising Compound A, or a pharmaceutically acceptable salt thereof, are described in WO2013/184757, which is hereby incorporated by reference in its entirety. Compound A and its preparation and suitable pharmaceutical formulations containing the same are disclosed in WO2013/184757, for example, at Example 5. Compound A, or its pharmaceutically acceptable salt, may be administered as an oral pharmaceutical composition in the form of a capsule formulation or a tablet. Pharmaceutically acceptable salts of Compound A include the mesylate salt and the hydrochloride salt thereof. Preferably the pharmaceutically acceptable salt is the mesylate salt.

Other third generation TKIs useful in the combinations described herein and in the dosage regimens described herein include osimertinib (AZD9291), olmutinib (BI 1482694/HM61713), ASP8273, PF-06747775 and avitinib.

### Raf Inhibitors

The preferred Raf inhibitor used in the pharmaceutical combination of the present invention is Compound B, or a pharmaceutically acceptable salt thereof. Compound B is a compound having the structure:

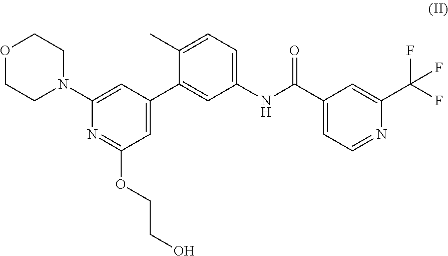 [SMILES: Cc1ccc(NC(=O)c2ccnc(C(F)(F)F)c2)cc1-c1cc(OCCO)nc(N2CCOCC2)c1]

Compound B, which is the compound of formula (II), is also known by the name of N-(3-(2-(2-hydroxyethoxy)-6-morpholinopyridin-4-yl)-4-methylphenyl)-2-(trifluoromethyl)isonicotinamide. Compound B is Example 1156 in published PCT application WO2014/151616, which is hereby incorporated by reference in its entirety. The preparation of Compound B, pharmaceutically acceptable salts of Compound B and pharmaceutical compositions comprising Compound B are also disclosed in the PCT application WO2014/151616, e.g., see pages 739-741. Compound B is an adenosine triphosphate (ATP)-competitive inhibitor of the v-raf Murine Sarcoma Viral Oncogene Homolog B1 (BRAF) and v-raf-1 Murine Leukemia Viral Oncogene Homolog 1 (CRAF) protein kinases. Compound B is a potent and selective Raf-inhibitor. It inhibits both BRAF and CRAF kinases, with similar sub-nanomolar potency, and inhibits the binding of only 2 other kinases to a similar degree out of 456 kinases tested (BRAF kinase IC₅₀=0.00073 μM and CRAF kinase IC₅₀=0.00020 μM).

Compound B has demonstrated efficacy in a wide range of MAPK pathway-driven human cancer cell lines and in vivo tumor xenografts including models harboring activating lesions in the KRAS, NRAS, and BRAF oncogenes.

In cell-based assays, Compound B demonstrated anti-proliferative activity in human cancer cell lines that contain a variety of mutations that activate MAPK signaling. For instance, Compound B inhibited the proliferation of melanoma models, including A-375 (BRAF V600E) and A-375 engineered to express BRAFi/MEKi resistance alleles, MEL-JUSO (NRAS Q61L), and IPC-298 (NRAS Q61L), as well as the non-small cell lung cancer cell line Calu-6 (KRAS Q61K) with IC₅₀ values ranging from 0.2-1.2 μM. In contrast, cell lines that have wild-type BRAF and RAS showed little response to Compound B with an IC₅₀ greater than 20 μM, suggesting selective activity in tumor cells with MAPK activation.

In vivo, treatment with Compound B generated tumor regression in several KRAS-mutant models including the NSCLC-derived Calu-6 (KRAS Q61K) and NCI-H358 (KRAS G12C) as well as the ovarian Hey-A8 (KRAS G12D, BRAF G464E) xenografts and in NRAS-mutant models including the SK-MEL-30 melanoma model. In all cases, anti-tumor effects were dose-dependent and well tolerated with minimal body weight loss.

As shown herein, preclinical data also demonstrated that the addition of Compound B to Compound A led to increased cell growth suppression compared to Compound A alone in a panel of EGFR mutant NSCLC cell lines.

Collectively, the in vitro and in vivo MAPK-pathway suppression and anti-proliferative activity observed for Compound B, as single agent and in the combination of the present invention, suggest that a Raf-inhibitor, e.g. Compound B, may be useful in the pharmaceutical combinations and dosing regimens described herein.

Unless otherwise specified, or clearly indicated by the text, or not applicable, reference to therapeutic agents useful in the COMBINATION OF THE INVENTION includes both the free base of the compounds, and all pharmaceutically acceptable salts of the compounds.

In one aspect, the present invention relates to the COMBINATION OF THE INVENTION for simultaneous, separate or sequential use.

In one aspect, the present invention relates to the COMBINATION OF THE INVENTION for use in the treatment of a cancer, particularly non-small cell lung cancer, more particularly EGFR mutant NSCLC.

The term “combination” or “pharmaceutical combination” is defined herein to refer to either a fixed combination in one dosage unit form, a non-fixed combination or a kit of parts for the combined administration where the therapeutic agents, e.g., the compound of formula (I) or a pharmaceutically acceptable salt thereof and the Raf inhibitor, may be administered together, independently at the same time or separately within time intervals, which preferably allows that the combination partners show a cooperative, e.g. synergistic effect.

The term “fixed combination” means that the therapeutic agents, e.g., the compound of formula I or a pharmaceutically acceptable salt thereof and the Raf inhibitor, are in the form of a single entity or dosage form.

The term “non-fixed combination” means that the therapeutic agents, e.g., the compound of formula (I) or a pharmaceutically acceptable salt thereof and the Raf inhibitor, are administered to a patient as separate entities or dosage forms either simultaneously, concurrently or sequentially with no specific time limits, wherein preferably such administration provides therapeutically effective levels of the two therapeutic agents in the body of the human in need thereof.

The term “synergistic effect” as used herein refers to action of two therapeutic agents such as, for example, (a) the compound of formula (I) or a pharmaceutically acceptable salt thereof, and (b) a Raf inhibitor, producing an effect, for example, delaying the symptomatic progression of a cancer, symptoms thereof, or overcoming resistance development or reversing the resistance acquired due to pre-treatment, which is greater than the simple addition of the effects of each therapeutic agent administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively. Synergy may be further shown by calculating the synergy score of the combination according to methods known by one of ordinary skill.

The term “pharmaceutically acceptable salt” refers to a salt that retains the biological effectiveness and properties of the compound and which typically is not biologically or otherwise undesirable. The compound may be capable of forming acid addition salts by virtue of the presence of an amino group.

The terms “a” and “an” and “the” and similar references in the context of describing the invention (especially in the context of the following claims) are to be construed to cover both the singular and the plural, unless otherwise indicated herein or clearly contradicted by context.

Where the plural form is used for compounds, salts, and the like, this is taken to mean also a single compound, salt, or the like.

The term “treating” or “treatment” is defined herein to refer to a treatment relieving, reducing or alleviating at least one symptom in a subject or affecting a delay of progression of a disease. For example, treatment can be the diminishment of one or several symptoms of a disease or complete eradication of a disease, such as cancer. Within the meaning of the present invention, the term “treat” also denotes to arrest, delay the progression and/or reduce the risk of developing resistance towards EGFR inhibitor treatment or otherwise worsening a disease.

The term “subject” or “patient” as used herein refers to a human suffering from a cancer, preferably lung cancer, e.g. NSCLC, in particular, EGFR mutant NSCLC.

The term “administration” is also intended to include treatment regimens in which the therapeutic agents are not necessarily administered by the same route of administration or at the same time.

The term “jointly therapeutically active” or “joint therapeutic effect” as used herein means that the therapeutic agents may be given separately (in a chronologically staggered manner, especially a sequence-specific manner) in such time intervals that they prefer, in a human subject to be treated, still show a beneficial (preferably synergistic) interaction (joint therapeutic effect). Whether this is the case can, inter alfa, be determined by following the blood levels, showing that both therapeutic agents are present in the blood of the human to be treated at least during certain time intervals.

The term “effective amount” or “therapeutically effective amount” of a combination of therapeutic agents is defined herein to refer to an amount sufficient to provide an observable improvement over the baseline clinically observable signs and symptoms of the cancer treated with the combination.

The term “about” refers to a statistically acceptable variation in a given value, and typically is +/−5% or 10% , On the other hand, when a numerical value is quoted without being accompanied by the term “about”, it will be understood that this numerical value will include a variation of that value which is statistically acceptable in the art.

The expression “until minimal residual disease is achieved” as used herein means until the tumor burden decrease is less than 5% between two assessments carried out at least one month apart. It is envisaged that the pharmaceutical combinations and the therapeutic regimens provided herein may be useful to patients who are TKI treatment naïve patients, i.e. patients who have not received any prior therapy for NSCLC, e.g. advanced NSCLC. It is also envisaged that these patients include third-generation EGFR TKI-naive patients.

Thus the present invention provides a combination as described herein for use in the first-line treatment of non-small cell lung cancer, including EGFR-mutant NSCLC.

Patients likely to benefit from the pharmaceutical combinations and the therapeutic regimens provided herein also include pre-treated patients, e.g. patients who have received prior treatment with a first-generation EGFR TKI and/or a second generation EGFR TKI.

Tumor evaluations and assessment of tumor burden can be made based on RECIST criteria (Therasse et al 2000), New Guidelines to Evaluate the Response to Treatment in Solid Tumors, Journal of National Cancer Institute, Vol. 92; 205-16 and revised RECIST guidelines (version 1.1) (Eisenhauer et al 2009) European Journal of Cancer; 45:228-247.

A number of response criteria such as the ones described in the Table below may be used to assess the response of the tumor to treatment.

### Response Criteria for Target Lesions

| Response Criteria | Evaluation of target lesions |
|---|---|
| Complete Response (CR): | Disappearance of all non-nodal |
| | target lesions. In addition, any |
| | pathological lymph nodes assigned |
| | as target lesions must have a |
| | reduction in short axis to <10 mm ¹ |
| Partial Response (PR): | At least a 30% decrease in the sum |
| | of diameter of all target lesions, |
| | taking as reference the baseline |
| | sum of diameters. |
| Progressive Disease (PD): | At least a 20% increase in the sum |
| | of diameter of all measured target |
| | lesions, taking as reference the smallest |
| | sum of diameter of all target |
| | lesions recorded at or after baseline. |
| | In addition to the relative increase |
| | of 20%, the sum must also demonstrate |
| | an absolute increase of at least |
| | 5 mm ². |
| Stable Disease (SD): | Neither sufficient shrinkage to qualify |
| | for PR or CR nor an increase in |
| | lesions which would qualify for PD. |
| Unknown (UNK) | Progression has not been documented |
| | and one or more target lesions |
| | have not been assessed or have been |
| | assessed using a different method |
| | than baseline.³ |

Tumor burden (also called “tumor load”) refers to the number of cancer cells, the size of a tumor, or the amount of cancer in the body. A subject suffering from cancer is defined to include as having progressed on, or no longer responding to therapy with one or more agents, or being intolerant to with one or more agents when the cancer he or she is suffering from, has progressed i.e. the tumor burden has increased. Progression of cancer such as NSCLC or tumors may be indicated by detection of new tumors or detection of metastasis or cessation of tumor shrinkage. The progression of cancer and the assessment of tumor burden increase or decrease may be monitored by methods well known to those in the art. For example, the progression may be monitored by way of visual inspection of the cancer, such as, by means of X-ray, CT scan or MRI or by tumor biomarker detection. An increased growth of the cancer may indicate progression of the cancer. Assessment of tumor burden assessment may be determined by the percent change from baseline in the sum of diameters of target lesions. Tumor burden assessment, whereby a decrease or increase in tumor burden is determined, will normally be carried out at various intervals, e.g. in successive assessments carried out at least 1, 2, 3 month(s), preferably one month apart.

The COMBINATION OF THE INVENTION is particularly useful for the treatment of a lung cancer. The lung cancer that may be treated by the COMBINATION OF THE INVENTION may be a non-small cell lung cancer (NSCLC). The most common types of NSCLC are squamous cell carcinoma, large cell carcinoma, and lung adenocarcinoma. Less common types of NSCLC include pleomorphic, carcinoid tumor, salivary gland sarcoma, and unclassified sarcoma. The NSCLC, and in particular lung adenocarcinoma, may be characterized by aberrant activation of EGFR, in particular amplification of EGFR, or somatic mutation of EGFR.

The lung cancer to be treated thus includes EGFR mutant NSCLC. It is envisaged that the combination of the present invention will be useful in treating advanced EGFR mutant NSCLC. Advanced NSCLC refers to patients with either locally advanced or metastatic NSCLC. Locally advanced NSCLC is defined as stage IIIB NSCLC not amenable to definitive multi-modality therapy including surgery. Metastatic NSCLC refers to stage IV NSCLC.

For the identification of EGFR mutant cancers that may be treated according to the methods described hereinEGFR mutation status may be determined by tests available in the art, e.g. QIAGEN therascreen® EGFR test or other FDA approved tests. The therascreen EGFR RGQ PCR Kit is an FDA-approved, qualitative real-time PCR assay for the detection of specific mutations in the EGFR oncogene. Evidence of EGFR mutation can be obtained from existing local data and testing of tumor samples. EGFR mutation status may be determined from any available tumor tissue.

The present invention relates to the COMBINATION OF THE INVENTION for use in the treatment of a cancer, particularly lung cancer, particularly non-small cell lung cancer (NSCLC), e.g. EGFR mutant NSCLC.

The cancer, particularly the lung cancer, more particularly the EGFR mutant non-small cell lung cancer (NSCLC) to be treated may harbor a mutation of EGFR C797, which is the binding site of EGF816 and other third-generation EGFR tyrosine kinase inhibitors.

A C797S mutation in EGFR (i.e. a single point mutation resulting in a cysteine to serine at position 797) has been observed clinically as a resistance mechanism in patients treated with osimertinib and in at least one patient treated with EGF816 so far. EGFR C797S mutation is hypothesized to disrupt binding to third-generation EGFR TKIs to EGFR, The C797S mutation may occur on a different EGFR allele to a T790M mutation, i.e. the EGFR mutant NSCLC may harbor a C797m/T790M in trans. If the C797S mutation occurs on the same allele of EGFR as the T790M mutation, the mutations are said to be in cis (C797m/T790M in cis).

The cancer, particularly the lung cancer, more particularly the non-small cell lung cancer (NSCLC) may also harbor an EGFR G719S mutation, EGFR G719C mutation, EGFR G719A mutation, EGFR L858R mutation, EGFR L861Q mutation, an EGFR exon 19 deletion, an EGFR exon 20 insertion, EGFR T790M mutation, EGFR T854A mutation, EGFR D76 1Y mutation, EGFR C797S mutation, or any combination thereof.

The present pharmaceutical combination of the invention may be particularly useful for treating NSCLC which harbors an EGFR L858R mutation, an EGFR exon 19 deletion or both. The NSCLC to be treated may also harbor a further EGFR T790M mutation which may be a de novo mutation or an acquired mutation. The acquired mutation may have arisen after treatment with a first-generation EGFR TKI (e.g. erlonitinib, gefitinib, icotinib, or any combination thereof) and/or treatment with a second generation TKI (e.g. afatinib, dacomitinib or both).

The present pharmaceutical combination of the invention may also be useful for patients who are treatment naïve with respect to a third generation TKI, for example osimertinib. Patients who may benefit from the combination therapy include those suffering from cancer, e.g. NSCLC, which also harbors EGFR C797m/T790M in cis (i.e. a C797 mutation and a T790M in cis). C797m is a mutation at EGFR C797 and confers resistance to EGF816 and other third-generation EGFR tyrosine kinase inhibitors. Additionally, these patients may also present tumors with an additional mutation selected from MET amplification, exon 14 skipping mutation, BRAF fusion or mutation, and any combination thereof.

In a preferred embodiment, the NSCLC to be treated carries an EGFR mutation which is selected from an EGFR exon 19 deletion, an EGFR T790M mutation or both an EGFR exon 19 deletion, an EGFR T790M; or from an EGFR L858R mutation or both an EGFR L858R and EGFR T790M.

In another embodiment, the present invention provides a COMBINATION OF THE INVENTION for the use of a cancer, particularly lung cancer, particularly non-small cell lung cancer (NSCLC), e.g. EGFR mutant NSCLC, characterized by harboring an EGFR C797S mutation.

In one embodiment, the present invention relates to the COMBINATION OF THE INVENTION for use in the treatment of a cancer, particularly lung cancer, particularly non-small cell lung cancer (NSCLC), e.g. EGFR mutant NSCLC, characterized by harboring an EGFR T790M mutation.

In one embodiment, EGFR T790M mutation is a de novo mutation. The term “de novo mutation” is defined herein to refer to an alteration in a gene that is detectable or detected in a human, before the onset of any treatment with an EGFR inhibitor. De novo mutation is a mutation which normally has occurred due to an error in the copying of genetic material or an error in cell division, e.g., de novo mutation may result from a mutation in a germ cell (egg or sperm) of one of the parents or in the fertilized egg itself, or from a mutation occurring in a somatic cell. A “de novo” T790M is defined as the presence of EGFR T790M mutation in NSCLC patients who have NOT been previously treated with any therapy known to inhibit EGFR.

In another embodiment, EGFR T790M mutation is an acquired mutation, e.g., a mutation that is not detectable or detected before the cancer treatment but become detectable or detected in the course of the cancer treatment, particularly treatment with one or more EGFR inhibitors, e.g., gefitinib, erlotinib, or afatinib.

In one embodiment, the present invention relates to the COMBINATION OF THE INVENTION for use in the treatment of a cancer, particularly lung cancer, particularly non-small cell lung cancer (NSCLC), e.g. EGFR mutant NSCLC, characterized by harboring EGFR T790M mutation in combination with any other mutation selected from the list consisting of EGFR C797S mutation, EGFR G719S mutation, EGFR G719C mutation, EGFR G719A mutation, EGFR L858R mutation, EGFR L861Q mutation, an EGFR exon 19 deletion, and an EGFR exon 20 insertion.

In one embodiment, the present invention relates to the COMBINATION OF THE INVENTION for use in the treatment of a cancer, particularly lung cancer, particularly non-small cell lung cancer (NSCLC), e.g. EGFR mutant NSCLC, characterized by harboring EGFR T790M mutation in combination with any other mutation selected from the list consisting of EGFR C797S mutation , EGFR G719S mutation, EGFR G719C mutation, EGFR G719A mutation, EGFR L858R mutation, EGFR L861Q mutation, an EGFR exon 19 deletion, and an EGFR exon 20 insertion, wherein EGFR T790M mutation is a de novo mutation.

In another embodiment, the present invention relates to the COMBINATION OF THE INVENTION for use in the treatment of a cancer, particularly lung cancer, particularly non-small cell lung cancer (NSCLC), e.g. EGFR mutant NSCLC, characterized by harboring EGFR T790M mutation in combination with any other mutation selected from the list consisting of EGFR C797S mutation, EGFR G719S mutation, EGFR G719C mutation, EGFR G719A mutation, EGFR L858R mutation, EGFR L861Q mutation, an EGFR exon 19 deletion, and an EGFR exon 20 insertion, wherein EGFR T790M mutation is an acquired mutation.

In one embodiment, the present invention relates to the COMBINATION OF THE INVENTION for use in the treatment of a cancer, particularly lung cancer, particularly non-small cell lung cancer (NSCLC), e.g. EGFR mutant NSCLC, characterized by harboring EGFR mutation selected from the group consisting of C797S, G719S, G719C, G719A, L858R, L861Q, an exon 19 deletion mutation, and an exon 20 insertion mutation. In a preferred embodiment, the present invention relates to the COMBINATION OF THE INVENTION for use in the treatment of a cancer characterized by harboring at least one of the following mutations: EGFR L858R and an EGFR exon 19 deletion.

In one embodiment, the present invention relates to the COMBINATION OF THE INVENTION for use in the treatment of a cancer, particularly lung cancer, particularly non-small cell lung cancer (NSCLC), e.g. EGFR mutant NSCLC, characterized by harboring EGFR mutation selected from the group consisting of C797S, G719S, G719C, G719A, L858R, L861Q, an exon 19 deletion mutation, and an exon 20 insertion mutation, and further characterized by harboring at least one further EGFR mutation selected from the group consisting of T790M, T854A and D761Y mutation.

In a preferred embodiment, the present invention relates to the COMBINATION OF THE INVENTION for use in the treatment of a cancer, particularly lung cancer, particularly non-small cell lung cancer (NSCLC), e.g. EGFR mutant NSCLC, characterized by harboring EGFR L858R mutation or EGFR exon 19 deletion, and further harboring an EGFR T790M mutation.

In one embodiment, the present invention relates to the COMBINATION OF THE INVENTION for use in the treatment of a cancer, particularly lung cancer, particularly non-small cell lung cancer (NSCLC), e.g. EGFR mutant NSCLC, wherein the cancer is resistant to a treatment with an EGFR tyrosine kinase inhibitor, or is developing a resistance to a treatment with an EGFR tyrosine kinase inhibitor, or is under high risk of developing a resistance to a treatment with an EGFR tyrosine kinase inhibitor. The EGFR tyrosine kinase inhibitor includes erlotinib, gefitinib, afatinib and osimertinib.

In another embodiment, the present invention relates to the COMBINATION OF THE INVENTION for use in the treatment of a cancer, particularly lung cancer, particularly non-small cell lung cancer (NSCLC), e.g. EGFR mutant NSCLC, wherein the cancer is resistant to a treatment with an EGFR tyrosine kinase inhibitor, or is developing a resistance to a treatment with an EGFR tyrosine kinase inhibitor, or is under high risk of developing a resistance to a treatment with an EGFR tyrosine kinase inhibitor, wherein the EGFR tyrosine kinase inhibitor is selected from the group consisting of erlotinib, gefitinib and afatinib.

The COMBINATION OF THE INVENTION is also suitable for the treatment of poor prognosis patients, especially such poor prognosis patients having a cancer, particularly lung cancer, particularly non-small cell lung cancer (NSCLC), e.g. EGFR mutant NSCLC, which becomes resistant to treatment employing an EGFR inhibitor, e.g. a cancer of such patients who initially had responded to treatment with an EGFR inhibitor and then relapsed. In a further example, said patient has not received treatment employing a Raf inhibitor. This cancer may have acquired resistance during prior treatment with one or more EGFR inhibitors. For example, the EGFR targeted therapy may comprise treatment with gefitinib, erlotinib, lapatinib, XL-647, HKI-272 (Neratinib), BIBW2992 (Afatinib), EKB-569 (Pelitinib), AV-412, canertinib, PF00299804, BMS 690514, HM781-36b, WZ4002, AP-26113, cetuximab, panitumumab, matuzumab, trastuzumab, pertuzumab, Compound A of the present invention, or a pharmaceutically acceptable salt thereof. In particular, the EGFR targeted therapy may comprise treatment with gefitinib, erlotinib, and afatinib. The mechanisms of acquired resistance include, but are not limited to, developing a second mutation in the EGFR gene itself, e.g. T790M, EGFR amplification; and/or FGFR deregulation, FGFR mutation, FGFR ligand mutation, FGFR amplification, MET amplification or FGFR ligand amplification. In one embodiment, the acquired resistance is characterized by the presence of T790M mutation in EGFR.

The COMBINATION OF THE INVENTION is also suitable for the treatment of patients having a cancer, particularly lung cancer, particularly non-small cell lung cancer (NSCLC), e.g. EGFR mutant NSCLC, wherein the cancer is developing resistance to treatment employing an EGFR inhibitor as a sole therapeutic agent. The EGFR inhibitor may be a first generation inhibitor (e.g. erlotinib, gefitinib and icotinib), a second generation inhibitor (e.g. afatinib and dacomitinib) or a third generation inhibitor (e.g. osimertinib or nazartinib).

The COMBINATION OF THE INVENTION is also suitable for the treatment of patients having a cancer, particularly lung cancer, particularly non-small cell lung cancer (NSCLC), e.g. EGFR mutant NSCLC, wherein the cancer is under a high risk of developing a resistance to a treatment with an EGFR inhibitor as a sole therapeutic agent. Since almost all cancer patients harboring EGFR mutations, in particular NSCLC patients, develop with time resistance to the treatment with such EGFR tyrosine kinase inhibitors as gefitinib, erlotinib, afatinib or osimertinib, a cancer of said patient is always under a high risk of developing a resistance to a treatment with an EGFR inhibitor as a sole therapeutic agent. And thus, cancers harboring EGFR C797S, EGFR G719S mutation, EGFR G719C mutation, EGFR G719A mutation, EGFR L858R mutation, EGFR L861Q mutation, an EGFR exon 19 deletion, an EGFR exon 20 insertion, EGFR T790M mutation, EGFR T854A mutation or EGFR D761Y mutation, or any combination thereof are under a high risk of developing a resistance to a treatment with an EGFR inhibitor as a sole therapeutic agent.

The combinations and therapeutic regimens provided herein may be suitable for:
treatment naive patients who have locally advanced or metastatic NSCLC with EGFR sensitizing mutation (e.g., L858R and/or ex19del);
patients who have locally advanced or metastatic NSCLC with EGFR sensitizing mutation and an acquired T790M mutation (e.g., L858R and/or ex19del, T790M+) following progression on prior treatment with a first-generation EGFR TKI or second-generation EGFR TKI: these patients include patients who have not received any agent targeting EGFR T790M mutation (i.e., third-generation EGFR TKI).
patients who have locally advanced or metastatic NSCLC with EGFR sensitizing mutation and a “de novo” T790M mutation (i.e., no prior treatment with any agent known to inhibit EGFR including EGFR TKI): these patients include patients who not have received any prior 3rd generation EGFR TKI.

Thus, the present invention includes a method of treating a patient having a cancer, specially a lung cancer (e.g. NSCLC) which comprises selectively administering a therapeutically effective amount of nazartinib, or a pharmaceutically acceptable salt thereof, and/or a therapeutically effective amount of the COMBINATION OF THE INVENTION to a patient having previously been determined to have a cancer, particularly lung cancer (e.g. NSCLC) which harbors one or more of the mutations described herein.

The present invention also relates to a method of treating a patient having a cancer, specially a lung cancer (e.g. NSCLC) which comprises:
(a) determining or having determined that the patient has a cancer which harbors one or more of the mutations described herein; and
(b) administering a therapeutically effective amount of nazartinib, or a pharmaceutically acceptable salt thereof, and/or a therapeutically effective amount of the COMBINATION OF THE INVENTION to said patient.

The present invention also relates to a method of treating a patient having a cancer, specially a lung cancer (e.g. NSCLC), comprising selecting a patient for treatment based on the patient having been previously determined to have one or more of the mutations described herein, and administering a therapeutically effective amount of nazartinib, or a pharmaceutically acceptable salt thereof, and/or a therapeutically effective amount of the COMBINATION OF THE INVENTION to said patient.

Included herein within the expression “one or more of the mutations described herein” are EGFR C797S mutation, EGFR G719S mutation, EGFR G719C mutation, EGFR G719A mutation, EGFR L858R mutation, EGFR L861Q mutation, an EGFR exon 19 deletion, an EGFR exon 20 insertion, EGFR T790M mutation, EGFR T854A mutation or EGFR D761Y mutation, or any combination thereof.

In another aspect, the present invention relates to the pharmaceutical composition comprising the COMBINATION OF THE INVENTION and at least one pharmaceutically acceptable carrier.

As used herein, the term “pharmaceutically acceptable carrier” includes generally recognized as safe for patients (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences). Except insofar as any conventional carrier is incompatible with Compound A or Compound B its use in the pharmaceutical compositions or medicaments is contemplated.

In another aspect, the present invention relates to use of Compound A or a pharmaceutical acceptable salt thereof for the preparation of a medicament for use in combination with a Raf inhibitor for the treatment of lung cancer. In another aspect, the present invention relates to use of a Raf inhibitor for the preparation of a medicament for use in combination with Compound A or a pharmaceutical acceptable salt thereof for the treatment of lung cancer, particularly non-small cell lung cancer (NSCLC), more particularly EGFR mutant NSCLC.

In another aspect, the present invention relates to a method of treating a lung cancer, particularly non-small cell lung cancer (NSCLC), e.g. EGFR mutant NSCLC, comprising simultaneously, separately or sequentially administering to a subject in need thereof the COMBINATION OF

THE INVENTION in a quantity which is jointly therapeutically effective against said lung cancer, particularly non-small cell lung cancer (NSCLC), e.g. EGFR mutant NSCLC.

### Dosages

The dosages or doses quoted herein, unless explicitly mentioned otherwise, refer to the amount present, in the drug product, of Compound A or of Compound B, calculated as the free base.

When Compound A is administered as monotherapy in the dosing regimen described herein, the dose of Compound A may be selected from a range of 50-350 mg, more preferably from a range of 50-150 mg. Compound A may be administered at a dosage of 50, 75, 100, 150, 200, 225, 250, 300 mg once daily. Thus, Compound A may be administered at a dosage of 50, 75, 100 or 150 mg once daily; more preferably, 50, 75 or 100 mg once daily. The 50, 75 or 100 mg doses may be better tolerated without loss of efficacy. In a preferred embodiment, Compound A may be administered at a dosage of 100 mg once daily.

When administered as part of the combination therapy, Compound A may be administered at a dosage of 25-150 mg, preferably 25-100 mg, preferably given once daily. In a preferred embodiment, Compound A may be administered at a dosage of 25, 50, 75, or 100 mg, e.g. once daily as part of the combination therapy. Preferably the dose is selected from 50, 75 and 100 mg of the drug substance referred to as its free base, as these doses may be better tolerated without loss of efficacy. In a preferred embodiment, Compound A is administered at a dosage of 100 mg once daily as part of the combination therapy.

The daily dose of Compound B may be selected from a range of 200 to 1200 mg, preferably from a range of 400-1200 mg, more preferably from a range of 400-800 mg. Compound B is preferably administered once daily. The dosage may be 200, 300, 400 mg or 800 mg of Compound B. The dosage may be preferably 200, 400 or 800 mg.

Some embodiments of the pharmaceutical combinations of the invention are enumerated below

| Dosage (mg), based on the | Dosages (mg), based on the |
|---|---|
| free base, of EGF816 | free base, of Compound B |
| 25 | 200, 300 or 400 |
| 50 | 200, 400 or 800 |
| 75 | 200, 400 or 800 |
| 100 | 200, 400 or 800 |
| 150 | 200, 400 or 800 |

The individual therapeutic agents of the COMBINATION OF THE INVENTION, i.e. the third generation EGFR inhibitor and the Raf inhibitor, may be administered separately at different times during the course of therapy or concurrently in divided or single combination forms. For example, the method of treating a cancer, particularly lung cancer, particularly non-small cell lung cancer (NSCLC), e.g. EGFR mutant NSCLC, according to the invention may comprise: (i) administration of Compound A in free or pharmaceutically acceptable salt form, and (ii) administration of a Raf inhibitor, preferably Compound B, in free or pharmaceutically acceptable salt form, simultaneously or sequentially in any order, in jointly therapeutically effective amounts e.g. in daily or intermittently dosages corresponding to the amounts described herein.

It can be shown by established test models that a COMBINATION OF THE INVENTION results in the beneficial effects described herein before. The person skilled in the art is fully enabled to select a relevant test model to prove such beneficial effects. The pharmacological activity of a COMBINATION OF THE INVENTION and/or of the dosing regimen described herein may, for example, be demonstrated in a clinical study or in an in vivo or in vitro test procedure as essentially described hereinafter.

In one important aspect, the present invention aims to provide a therapy with clinical benefit compared to a single agent third generation EGFR inhibitor, or compared with the second combination partner, with the potential to prevent or delay the emergence of treatment-resistant disease.

The present inventors have observed that clinical responses to first/ second generation EGFR TKIs in the 1st-line setting and to EGF816 in EGFR T790M-mutant NSCLC in the second-line and beyond are generally characterized by rapid acquisition of maximal tumor response, followed by a prolonged period of relatively stable disease control. During this period of stable disease control, there is a state of minimal residual disease, wherein the tumor tissue remains relatively dormant prior to the outgrowth of drug-resistant clone(s). It is envisaged that once this tumor shrinkage plateau is achieved, the administration of a combination of a third generation EGFR inhibitor and a Raf inhibitor will be especially beneficial in the treatment of the cancer. The combination add-on therapy on top of the single agent therapy would be beneficial in targeting viable “persister” tumor cells and thus may prevent the emergence of drug-resistant clone(s).

The present invention thus provides a dosing regimen which takes advantage of the initial efficacy of the EGFR inhibitor, suitably the third-generation EGFR inhibitor, and the advantageous effects of the combination of the invention.

The present invention provides a method for treating EGFR mutant lung cancer in a human in need thereof, particularly EGFR mutant NSCLC, comprising
(a) administering a therapeutically effective amount of a third-generation EFGR inhibitor (such as Compound A, or a pharmaceutically acceptable salt thereof as monotherapy until minimal residual disease is achieved (i.e., until the tumor burden decrease is less than 5% between two assessments carried out at least one month apart); followed by
(b) administering a therapeutically effective amount of a pharmaceutical combination of Compound A, or a pharmaceutically acceptable salt thereof, and a Raf inhibitor, particularly, Compound B or a pharmaceutically acceptable salt thereof.

The present invention provides Compound A, or a pharmaceutically acceptable salt thereof, for use in treating EGFR mutant lung cancer in a human in need thereof, particularly EGFR mutant NSCLC, wherein

(a) Compound A, or a pharmaceutically acceptable salt thereof is administered as monotherapy until minimal residual disease is achieved (i.e., until the tumor burden decrease is less than 5% between two assessments carried out at least one month apart); and

(b) a pharmaceutical combination of Compound A, or a pharmaceutically acceptable salt thereof, and a Raf inhibitor, particularly, Compound B or a pharmaceutically acceptable salt thereof, is thereafter administered.

The progression of cancer, tumor burden increase or decrease, and response to treatment with an EGFR inhibitor may be monitored by methods well known to those in the art. Thus the progression and the response to treatment may be monitored by way of visual inspection of the cancer, such as, by means of X-ray, CT scan or MRI or by tumor biomarker detection. For example, an increased growth of the cancer indicates progression of the cancer and lack of response to the therapy. Progression of cancer such as NSCLC or tumors may be indicated by detection of new tumors or detection of metastasis or cessation of tumor shrinkage. Tumor evaluations, including assessments of tumor burden decrease or tumor burden increase, can be made based on RECIST criteria (Therasse et al 2000), New Guidelines to Evaluate the Response to Treatment in Solid Tumors, Journal of National Cancer Institute, Vol. 92; 205-16 and revised RECIST guidelines (version 1.1) (Eisenhauer et al 2009) European Journal of Cancer; 45:228-247. Tumor progression may be determined by comparison of tumor status between time points after treatment has commenced or by comparison of tumor status between a time point after treatment has commenced to a time point prior to initiation of the relevant treatment.

Determination of the attainment of the state of minimal residual disease or stable disease response may thus be determined by using Response Evaluation Criteria In Solid Tumors (RECIST 1.1) or WHO criteria. A stable disease (Stable Disease or SD) response may be defined as a response where the target lesions show neither sufficient shrinkage to qualify for Partial Response (PR) nor sufficient increase to qualify for Progressive Disease (PD), taking as reference the smallest sum Longest Diameter (LD) of the target lesions since the treatment started. Other Response Criteria may be defined as follows.

Complete Response (CR): Disappearance of all target lesions

Partial Response (PR): At least a 30% decrease in the sum of the LD of target lesions, taking as reference the baseline sum LD.

Progressive Disease (PD): At least a 20% increase in the sum of the LD of target lesions, taking as reference the smallest sum LD recorded since the treatment started or the appearance of one or more new lesions.

The treatment period during which the EGFR inhibitor as monotherapy is administered is a period of time sufficient to achieve minimal residual disease may thus be readily measured by the skilled person in the art. The treatment period may consist of one, two, three, four, five, six or more 28-day cycles, preferably two or three cycles.

In another aspect, the present invention relates to a commercial package comprising the COMBINATION OF THE INVENTION and instructions for simultaneous, separate or sequential administration of the COMBINATION OF THE INVENTION to a patient in need thereof. In one embodiment, the present invention provides a commercial package comprising the third generation EGFR inhibitor Compound A, or a pharmaceutically acceptable salt thereof, and instructions for the simultaneous, separate or sequential use with a Raf inhibitor, preferably Compound B or a pharmaceutically acceptable salt thereof, for use in the treatment of a cancer, particularly lung cancer, particularly non-small cell lung cancer (NSCLC), e.g. EGFR mutant NSCLC, and preferably wherein the cancer is characterized by a mutant EGFR; for example, wherein the mutant EGFR comprises C797S, G719S, G719C, G719A, L858R, L861Q, an exon 19 deletion mutation, an exon 20 insertion mutation, EGFR T790M, T854A or D761Y mutation, or any combination thereof, and preferably wherein said cancer has acquired resistance during prior treatment with one or more EGFR inhibitors or developing a resistance to a treatment with one or more EGFR inhibitors, or under high risk of developing a resistance to a treatment with an EGFR inhibitor.

The following Examples illustrate the invention described above, but are not, however, intended to limit the scope of the invention in any way. Other test models known to the person skilled in the pertinent art can also determine the beneficial effects of the claimed invention.

### EXAMPLES

### Example 1: Short-Term Viability Assays: Compound B Enhances the Efficacy of EGF816 (Compound A)

The potential efficacy of adding a MAPK pathway inhibitor such as Compound B to a third-generation EGFR tyrosine kinase inhibitor such as EGF816 in EGFR mutant NSCLC was assessed as follows. A panel of EGFR mutant NSCLC cell lines was treated with a fixed dose (300 nM) of

EGF816 (“Compound A”) or DMSO in combination with Compound B across a 10-dose range for 5 days.

### Methods

Cell lines:

PC9, HCC827, HCC4006, NCI-H1975 and MGH707 are all EGFR mutant NSCLC cell lines sensitive to EGF816. PC9, HCC827, HCC4006 and NCI-H1975 were obtained from the cancer cell line encyclopedia (CCLE) database. MGH707 were obtained from Massachusetts General Hospital. All cell lines were maintained in RMPI media supplemented with 10% fetal bovine serum.

Compounds:

Compound A (EGF816) and Compound B were all re-suspended in DMSO at a concentration of 10 mM for a stock solution and further diluted for the experiments as indicated.

### Experimental Procedure

The following EGFR mutant (EGFR mt) NSCLC cell lines were plated at the following densities: HCC827 (exon 19 deletion, or ex19del for short) (500/well), HCC4006 (ex19del) (500/well), PC9 (ex19del)(500/well), and MGH707 parental (1000/well) into white 384-well plates (#3707, Corning, Oneonta, N.Y., USA) and stored in an incubator overnight at 37° C., 95% relative humidity, and 5% CO₂. Compounds were serial diluted (1:3 dilution) in DMSO in an acoustic plate (#P-05525, Labcyte, San Jose, Calif., USA) using a Biomek liquid handler (Beckman, Indianapolis, Ind., USA). Compound A was dispensed into the first row of a Labcyte P-05525 source plate. The compound plates were then used to deliver combinations to assay plates using an acoustic dispenser (Echo-555, Labcyte, San Jose, Calif., USA). Each dispense was 50 nL which achieved a 1:1000 dilution (ie. 50 nL dispense of a 10 mM compound becomes 10 uM in 50 uL of cell solution). The combination treatment included a second dispense of 50 nL of Compound A to achieve a final concentration of 0.3 uM. Upon completion of dosing each assay plate was returned to an incubator (37° C., 95% relative humidity, and 5% CO₂). 25 uL/well of CellTiter-Glo One Solution cell viability reagent (#G8462, Promega, Madison, Wis., USA) was added to an untreated plate of each cell line using a bulk dispenser (EL406, Biotek, Winooski, Vt., USA) and after 20 minutes of incubation at room temperature, the plates were read on a microplate reader (Envision, Perkin Elmer, Hopkington, Mass., USA). These data are used to determine a baseline reading of cell number (Day 0) to assess cell growth, cell stasis, or cell death over the treatment time period. After 5 days of incubation, the assay plates are read using the same CellTiter-Glo assay reagent and read on the Envision microplate reader.

### Results

As can be seen from FIG. 1A and FIG. 1B, the growth of these EGFR mutant cell lines was suppressed by Compound B single agent (top curves of FIG. 1A and FIG. 1B). The presence of EGF816 alone leads to robust growth suppression in all of these cell lines (see decrease in activity from values with DMSO to values obtained with EGF816 at zero concentration of Compound B). When EGF816 is also present, the addition of Compound B leads to enhanced suppression of cell growth in the HCC4006, HCC827, and PC9 cell lines, but was less effective in the MGH707 model (bottom curves of FIG. 1A and FIG. 1B). Moreover, the addition of EGF816 led to a sensitization of these cells (HCC4006, HCC827 and PC9) to a Raf inhibitor such as Compound B, as they were active at lower doses when EGF816 was also present, as compared to single agent.

### Example 2: Long-Term Viability Assays Demonstrate That Combinations of a Third-Generation EGFR Tyrosine Kinase Inhibitor and a Raf-Inhibtor Slow the Outgrowth of Drug-Tolerant Cells

Combinations of Compound A and Compound B were further examined in long-term drug combination growth assays. The same EGFR mutant NSCLC cell lines as were used in Example 1 above were treated with EGF816 alone or EGF816 in combination with Compound B across a 5-dose range for 14 days as follows.

### Methods

PC9 (6000/well), HCC827 (4000/well), HCC4006 (5000/well), and MGH707 (5000/well) cells were plated into 96-well plates and the following day treated with EGF816 (300 nM) +either DMSO or a range of doses of either Compound B (0.03, 0.1, 0.3, 1 and 3 uM) for two weeks. Drug was refreshed twice per week. Cell confluence was used as a surrogate for cell number and was measured by an incucyte zoom at t=0, 4, 7, 10 and 14 days treatment.

### Results

As can be seen from FIG. 2, Compound B markedly suppressed the slow residual outgrowth of the EGFR mutant persister cells. In the case of the PC9 cell line, which is more exquisitely sensitive to single agent EGF816, combinations with Compound B led to a more pronounced drop in cell number. Again, the MGH707 model was most refractory to the combination of EGFR inhibitor and Raf-inhibitor.

### Conclusion and Discussion

The addition of the Raf inhibitor Compound B to EGF816 led to increased cell growth suppression compared to EGF816 alone in both short and more long-term assays in the majority of models tested. This is particularly impressive given that the dose of single agent EGF816 in these cells in the context of these experiments led to robust growth suppression and apoptosis, leaving minimal room for improvement. Taken together, Example 1 and Example 2 indicate that the combination of EGF816 with Compound B may have enhanced efficacy in the clinic compared to single agent EGF816. Targeting drug-tolerant cells with the present pharmaceutical combinations of the invention may thus be beneficial in improving the overall response and outcome for EGFR mutant NSCLC patients.

### Example 3: Phase Ib, Open-Label, Dose Escalation and/or Dose Expansion Study of EGF816 in Combination with Compound B in Patients with EGFR-Mutant NSCLC

Eligible patients for this study are patients who have advanced EGFR-mutant NSCLC, a disease that is currently incurable with any therapy. Treatment with EGF816 (Compound A) as a single-agent in either 1st line, treatment-naïve patients or in patients with acquired EGFR T790M gatekeeper mutations and/or who are naive to prior 3rd generation EGFR TKI is expected to lead to clinical benefit in the majority of patients. However, all patients are expected to develop treatment resistance and ultimate disease progression after a period of time on single agent EGF816.

Compound B is expected to be active in tumors in which signaling from BRAF or upstream (including activated RTK and Ras signaling) drives resistance or tumor cell persistence in the context of EGF816 treatment. As shown above, preclinical experiments demonstrated enhanced efficacy between EGF816 and Compound B in the impairment of proliferation/viability in EGFR-mutant NSCLC cells.

Because it is an inhibitor of CYP3A4/5, Compound B has the potential to increase exposure of EGF816 when administered together.

This study thus has a sound rationale supporting its potential to improve the clinical efficacy of EGF816. The potential benefit of this study is improved clinical benefit compared to a single agent EGFR TKI, with the potential to prevent or delay the emergence of treatment-resistant disease.

Study Design

This is a Phase Ib, open label, non-randomized dose escalation study of EGF816 in combination with Compound B followed by dose expansion of EGF816 in combination with Compound B in adult patients with advanced EGFR-mutant NSCLC. Patients must be either treatment-naïve in the advanced setting and harbor a sensitizing mutation in EGFR (ex19del or L858R) or have progressed on a 1st or 2nd generation EGFR TKI (e.g., erlotinib, gefitinib, afatinib) and harbor an EGFR T790M mutation within the tumor. Patients should not have previously received a 3^{rd} generation EGFR TKI (e.g., osimertinib, rociletinib, ASP8273).

Inclusion Criteria

Patients eligible for inclusion in this study must meet the following criteria:
Patient (male or female)≥18 years of age.
Patients must have histologically or cytologically confirmed locally advanced (stage IIIB) or metastatic (stage IV) EGFR mutant (ex19del, L858R) NSCLC.
Requirements of EGFR mutation status and prior lines of treatment:
   Treatment naive patients, who have locally advanced or metastatic NSCLC with EGFR sensitizing mutation (e.g., L858R and/or ex19del), have not received any systemic antineoplastic therapy for advanced NSCLC and are eligible to receive EGFR TKI treatment. Patients with EGFR exon 20 insertion/duplication are not eligible. Note: patients who have received only one cycle of chemotherapy in the advanced setting are allowed.
   Patients who have locally advanced or metastatic NSCLC with EGFR sensitizing mutation AND an acquired T790M mutation (e.g., L858R and/or ex19del, T790M+) following progression on prior treatment with a lst-generation EGFR TKI (e.g. erlotinib, gefitinib or icotinib) or 2nd-generation EGFR TKI (e.g., afatinib or dacomitinib). These patients may not have received more than 4 prior lines of antineoplastic therapy in the advanced setting, including EGFR TKI, and may not have received any agent targeting EGFR T790M mutation (i.e. 3rd-generation EGFR TKI). EGFR mutation testing must be performed after progression on EGFR TKI.
   Patients who have locally advanced or metastatic NSCLC with EGFR sensitizing mutation and a “de novo” T790M mutation (i.e. no prior treatment with any agent known to inhibit EGFR including EGFR TKI). These patients may not have received more than 3 prior lines of antineoplastic therapy in the advanced setting, and may not have received any prior 3rd generation EGFR TKI.

ECOG performance status: 0-1

All patients in both the escalation and expansion parts receive EGF816 100 mg qd as a single agent for approximately five 28-day cycles (Treatment period 1), and then receive EGF816 100 mg qd in combination with Compound B (Treatment period 2).

Assignment to the combination treatment is based in part on results of targeted genomic profiling of a tumor sample and cfDNA collected after approximately 4 cycles of EGF816 treatment.

Patients receiving the combination treatment also include patients with tumors characterized by EGFR C797 mutation and/T790M in cis. Also included are patients with tumors characterized by C797 mutation and T790M in cis, which also show MET amplification or exon 14 skipping mutation and/or BRAF fusion or mutation. C797 mutation is a direct resistance mechanism to the mode of action of EGF816. Compound B may block signalling downstream of activated EGFR in addition to blocking signalling from activated BRAF. Thus Compound B as the combination partner is expected to be useful therapy for such patients.

Efficacy assessments are performed at baseline and every 8 weeks (every 2 cycles) during treatment. Thus at least two post-baseline efficacy assessments will have been obtained before the patient starts the combination treatment. Patients who experience disease progression prior to the start of combination treatment are discontinued from the study, unless an exception is made for patients experiencing clinical benefit.

Starting Dose

| Study treatments | Dose | Frequency and/or Regimen |
|---|---|---|
| EGF816 | Starting dose: 100 mg | QD* |
| Compound B | Starting dose: 400 mg | QD |

*“QD” or “qd” means once daily

The daily dose of Compound A may also be selected from 25, 50, 75, 100, or 150 mg.

For this combination study, EGF816 (Compound A) is administered 100 mg qd (tablet; with or without food) on a continuous daily dosing schedule. In a previous study, overall response rates to EGF816 were found similar at 100 mg daily and 150 mg daily, but lower rates of rash and diarrhoea were observed at 100 mg daily. Therefore the 100 mg daily dose of EGF816 is chosen at first, as it is anticipated to be better tolerated than the 150 mg, particularly if the combination results in overlapping toxicity, while maintaining efficacy against EGFR-mutant NSCLC. The 100 mg qd dose is expected to provide a sufficiently large margin of tolerability for combinations in which drug-drug interaction may increase the exposure of EGF816 at 100 mg qd, compared to single agent EGF816. Based on PK data from the first cohort(s) of the combination for which the recommended regimen remains to be determined, the EGF816 dose may be decreased in combinations that result in an increase in EGF816 exposure, to maintain its exposure close to that of EGF816 single agent at 100 mg qd.

The starting dose of Compound B is 400 mg q.d. (tablet; preferably without food, on an empty stomach) on a continuous dosing schedule and may escalate to 800 mg qd. EGF816 is not predicted to affect the exposure Compound B.

The proposed starting regimen for EGF816 in combination with Compound B is EGF816 100 mg and Compound B 400 mg, taken together and each administered continuously once daily. Based on these prior safety data and the assumptions for Drug-Drug Interaction (DDI), the starting dose combination satisfies the EWOC criteria within BLRM.

Continuous dosing means administering of the agent without interruption for the duration of the treatment cycle. Continuous once daily administration thus refers to the administration of the therapeutic agent once daily with no drug holiday for the given treatment period.

The design of the dose escalation part of the study is chosen in order to characterize the safety and tolerability of Compound A in combination with Compound B in patients with EGFR-mutant NSCLC, and to determine a recommended dose and regimen. Where necessary, the dose escalation allows the establishment of the MTD (Maximum Tolerated Dose) of Compound A in combination with Compound B and will be guided by a Bayesian Logistic Regression Model (BLRM).

BLRM is a well-established method to estimate the Maximum Tolerated Dose (MTD) in cancer patients. The adaptive BLRM will be guided by the escalation with overdose control (EWOC) principle to control the risk of Dose Limiting Toxicity (DLT) in future patients on study. The use of Bayesian response adaptive models for small datasets has been accepted by EMEA (“Guideline on clinical trials in small populations”, Feb. 1, 2007) and endorsed by numerous publications (Babb et al 1998, Neuenschwander et al 2008, Neuenschwander et al 2010), and its development and appropriate use is one aspect of the FDA's Critical Path Initiative.

Selected Dose Levels

The selection of EGF816 dose level (100, 75, or 50 mg) for subsequent combination cohorts will depend on the EGF816 PK of earlier combination cohort(s).

TABLE
Provisional dose levels Compound B

| Dose | Proposed | Increment from previous |
|---|---|---|
| level | daily dose* | dose |
| −1** | 200 mg | −50% |
| 1 | 400 mg | (starting dose) |
| 2 | 800 mg | 100% |

*It is possible for additional and/or intermediate dose levels to be added during the course of the study Cohorts may be added at any dose level below the MTD in order to better understand safety, PK or PD.
**Dose level −1 represent treatment doses for patients requiring a dose reduction from the starting dose level. No dose reduction below dose level −1 is permitted for this study.

Treatment Duration:

Patients continue to receive the assigned treatment until disease progression by RECIST 1.1, unacceptable toxicity, start of a new anti-neoplastic therapy, discontinuation at the discretion of the investigator or patient, lost to follow-up, death, or termination of the study.

Objectives and related endpoints of this study:

| Objective | Endpoint |
|---|---|
| Primary | |
| To characterize the safety and | Safety: |
| tolerability of EGF816 in | Incidence of DLTs in first |
| combination with Compound B in | cycle of combination |
| patients with advanced EGFR- | (Dose escalation only) |
| mutant NSCLC in 1st line or ≥ 2nd | Incidence and severity of |
| line T790M+, 3rd gen EGFR TKI- | adverse events (AEs) and |
| naive and identify a recommended | serious adverse events |
| dose and regimen. | (SAEs), changes in |
| | hematology and chemistry |
| | values, vital signs, |
| | electrocardiograms (ECGs) |
| | graded as per NCI |
| | CTCAE version 4.03 |
| | (all patients) |
| | Tolerability: |
| | Dose interruptions, reductions |
| | and dose intensity |
| To estimate the preliminary anti- | Modified objective response |
| tumor activity of the addition of | rate (ORR2) per RECIST |
| Compound B in patients with | v1.1 (taking as baseline the |
| advanced EGFR-mutant NSCLC in | most recent assessment |
| 1st line or ≥ 2nd line T790M+, 3rd | prior to initiating combination) |
| gen EGFR TKI-naïve. | |
| Secondary | |
| To assess the preliminary anti- | Overall Response Rate |
| tumor activity of EGF816 single | (ORR), Progression-free |
| agent given for 5 cycles followed | survival (PFS), disease |
| by the addition of Compound B to | control rate (DCR), time to |
| EGF816 in advanced EGFR-mutant | response (TTR) and duration |
| NSCLC in 1st line or 2nd line and | of response (DOR) in |
| beyond T790M+, 3rd gen EGFR | accordance with Response |
| TKI-naive (endpoints ORR, PFS, | Evaluation Criteria in Solid |
| DOR, DCR). | Tumors (RECIST) v1.1 |
| To characterize the PK properties of | Plasma concentration vs. |
| EGF816 and Compound B. | time profiles; plasma PK |
| | parameters of EGF816 and |
| | Compound B |

@Partial Response (PR), Complete Response (CR), Stable Disease (SD)
*ORR is defined as proportion of patients with best overall response of PR + CR per RECIST v1.1 in the entire treatment period (from the beginning of EGF816 monotherapy to the end of the study treatment treatment), using pre-enrollment tumor assessment as baseline. ORR2 is defined as proportion of patients with best overall response of PR + CR per RECIST v1.1, using as baseline the latest tumor assessment prior to the start of combination treatment;
DOR is defined as the time from first documented response (PR or CR) to the date of first documented disease progression or death due to any cause; DCR is defined as the proportion of patients with best overall response of CR, PR, or SD;
PFS is defined as the time from the date of first dose of study treatment to the date of first documented disease progression (per RECIST v1.1) or death due to any cause.

1. A pharmaceutical combination of a third-generation EGFR tyrosine kinase inhibitor (TKI) and a Raf inhibitor.

2. The pharmaceutical combination according to claim 1, wherein the third-generation EGFR tyrosine kinase inhibitor is nazartinib, which is the compound of formula (I) 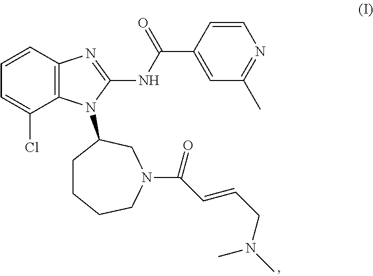 [SMILES: Cc1cc(C(=O)Nc2nc3cccc(Cl)c3n2[C@@H]2CCCCN(C(=O)/C=C/CN(C)C)C2)ccn1]
or a pharmaceutically acceptable salt thereof.

3. The pharmaceutical combination according to claim 1, wherein the Raf inhibitor is the compound of formula (II) (Compound B), 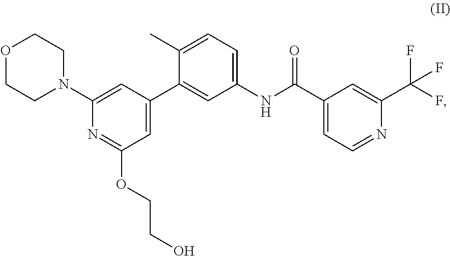 [SMILES: Cc1ccc(NC(=O)c2ccnc(C(F)(F)F)c2)cc1-c1cc(OCCO)nc(N2CCOCC2)c1]
or a pharmaceutically acceptable salt form.

4. The pharmaceutical combination according to claim 2, wherein the pharmaceutically acceptable salt of the compound of formula (I) is the mesylate salt or the hydrochloride salt.

5. The pharmaceutical combination according to claim 1 for simultaneous, separate or sequential use.

6. The pharmaceutical combination according to claim 1 for use in the treatment of a cancer in a patient.

7. The pharmaceutical combination for use according to claim 6, wherein the cancer is a lung cancer.

8. The pharmaceutical combination for use according to claim 7, wherein the lung cancer is a non-small cell lung cancer (NSCLC).

9. The pharmaceutical combination for use according to claim 6, wherein the cancer characterized by aberrant activation of EGFR or somatic mutation of EGFR.

10. The pharmaceutical combination for use according to claim 6 wherein the patient is suffering from the cancer is a treatment naïve patient.

11. The pharmaceutical combination for use according to claim 6 wherein the patient suffering from the cancer has received prior therapy with a tyrosine kinase inhibitor.

12. The pharmaceutical combination for use according to claim 6, wherein the cancer is resistant to treatment with an EGFR tyrosine kinase inhibitor, or developing resistance to treatment with an EGFR tyrosine kinase inhibitor, or under high risk of developing resistance to treatment with an EGFR tyrosine kinase inhibitor.

13. The pharmaceutical combination for use according claim 6, wherein the cancer is characterized by harboring EGFR G719S mutation, EGFR G719C mutation, EGFR G719A mutation, EGFR L858R mutation, EGFR L861Q mutation, an EGFR exon 19 deletion, an EGFR exon 20 insertion, EGFR T790M mutation, EGFR T854A mutation or EGFR 0761Y mutation, or any combination thereof.

14. The pharmaceutical combination for use according to claim 6, wherein the cancer is NSCLC and wherein the NSCLC harbors an EGFR L858R mutation, an EGFR exon 19 deletion or both.

15. The pharmaceutical combination for use according to claim 14, wherein the NSCLC further harbors an EGFR T790M mutation.

16. The pharmaceutical combination for use according to claim 15, wherein the EGFR T790M mutation is a de novo mutation.

17. The pharmaceutical combination for use according to claim 15, wherein the EGFR 1790M mutation is an acquired mutation.

18. The pharmaceutical combination for use according to claim 11, wherein the cancer has progressed after treatment with a first-generation EGFR TKI selected from erlotiinib, qeffitinib or icotinib and/or treatment with a second generation TKI selected from afatinib or dacomitin.

19. (canceled)

20. The pharmaceutical combination for use according to claim 6 wherein the cancer is characterized by EGFR C797 mutation and T790M in cis.

21. The pharmaceutical combination for use according to claim 20 wherein the cancer is further characterized by with MET amplification or exon 14 skipping mutation and/or BRAF fusion or mutation.

22.-23. (canceled)

24. A method of treating lung cancer comprising simultaneously, separately or sequentially administering to a subject in need thereof the pharmaceutical combination according to claim 1 in a quantity which is jointly therapeutically effective against said lung cancer.

25. A commercial package for use in the treatment of lung cancer comprising the pharmaceutical combination according to claim 1 and instructions for simultaneous, separate or sequential administration of said pharmaceutical combination to a human patient in need thereof.

26. A method for treating EGFR mutant lung cancer in a human in need thereof, particularly EGFR mutant NSCLC, comprising
(a) administering a therapeutically effective amount of a third-generation EFGR tyrosine kinase inhibitor selected frokm nazartinib, or a pharmaceutically acceptable salt thereof as monotherapy until the tumor burden decrease is less than 5% between two assessments carried out at least one month apart; followed by
(b) administering a therapeutically effective amount of a pharmaceutical combination of the third-generation EGFR tyrosine kinase inhibitor selected from nazartinib, or a pharmaceutically acceptable salt thereof and a Raf inhibitor selected from N-(3-(2-(2-hydroxyethoxy)-6-morpholinopyridin-4-yl)-4-methylphenyl)-2-(trifluoromethyl)isonicotinamide or a pharmaceutically acceptable salt thereof.

27. A compound that is Nazartinib, or a pharmaceutically acceptable salt thereof, for use in treating EGFR mutant lung cancer, particularly EGFR mutant NSCLC, wherein
(a) said nazartinib, or a pharmaceutically acceptable salt thereof, is administered as monotherapy until minimal residual disease is achieved; and
(b) a pharmaceutical combination of nazartinib, or a pharmaceutically acceptable salt thereof, and a Raf inhibitor selected from N-(3-(2-(2-hydroxyethoxy)-6-morpholinopyridin-4-yl)-4-methylphenyl)-2-(trifluoromethyl)isonicotinamide or a pharmaceutically acceptable salt thereof, is thereafter administered.

28. Nazartinib, or a pharmaceutically acceptable salt thereof, for use in treating EGFR mutant lung cancer, particularly EGFR mutant NSCLC, wherein
(a) nazartinib, or a pharmaceutically acceptable salt thereof, is administered as monotherapy until the tumor burden decrease of the patient suffering from said disease is less than 5% between two assessments carried out at least one month apart; and
(b) a pharmaceutical combination of bazartinib, or a pharmaceutically acceptable salt thereof, and (3 (2 (2-hydroxyethoxy)-6-morpholinopyridin-4-yl)-4-methylphenyl)-2-(trifluoromethyl)isonicotinamide, or a pharmaceutically acceptable salt thereof, is thereafter administered.

29. A third generation EGFR tyrosine kinase inhibitor, particularly nazartinib, or a pharmaceutically acceptable salt thereof, for use in a combination therapy with a Raf inhibitor, particularly the compound of formula (IL), or a pharmaceutically acceptable salt thereof, for the treatment of NSCLC.

30. A Raf inhibitor, particularly the compound of formula (II), or a pharmaceutically acceptable salt thereof, for use in a combination therapy with a third generation EGFR tyrosine kinase inhibitor, particularly nazartinib, or a pharmaceutically acceptable salt thereof, for the treatment of NSCLC.
